(19) United States
(12) Patent Application Publication
(10) Pub. No.: US 2020/0275685 A1
(43) Pub. Date: Sep. 3, 2020

(54) ORAL ADMINISTRATION FORM COMPRISING AN IRON-COPPER MIXTURE AND ITS USE AS A FOOD SUPPLEMENT

(71) Applicant: PIZETA PHARMA S.p.A., Visso (MC) (IT)

(72) Inventors: Maurizio Danilo Catanese, Visso (MC) (IT); Giulia Dal Moro, Visso (MC) (IT)

(73) Assignee: PIZETA PHARMA S.p.A., Visso (MC) (IT)

(21) Appl. No.: 16/764,256

(22) PCT Filed: Nov. 13, 2018
(86) PCT No.: PCT/IB2018/058919

§ 371 Date: May 14, 2020

(30) Foreign Application Priority Data
Nov. 14, 2017 (IT) 102017000130043

(51) Int. Cl. A23L 29/00; A23L 33/15; A61K 33/26

(52) U.S. Cl. CPC A23L 29/045; A23L 33/15; A61K 9/0053; A61K 33/26; A23L 29/035

(57) ABSTRACT

The present invention discloses an oral administration form comprising an association of Fe(II) and Cu(II). The oral administration form finds advantageous application as a food supplement for the supplementation of iron and copper, without side effects for the stomach and intestine.

### FIELD OF THE INVENTION

The present invention relates to an oral administration form comprising an association of Fe(II) and Cu(II). The oral administration form finds advantageous application as a food supplement for the supplementation of iron and copper, without side effects for the stomach and intestine.

### BACKGROUND ART

Iron-deficiency anaemia or sideropenia is a condition in which the body's total iron level is reduced considerably, thus compromising the iron supply to all tissues, but in particular to erythropoiesis.

Iron-deficiency anaemia is the most common form of anaemia worldwide. The causes of iron-deficiency are well known. Some are physiological, such as the increased need for iron in childhood, during body growth, in the third trimester of pregnancy, or during breastfeeding. Others include insufficient dietary intake, reduced intestinal absorption, or chronic blood loss. Blood loss may occur for different reasons: excessive menstrual loss is to be considered in women of childbearing age, metrorrhagia, and repeated pregnancies not sufficiently offset by martial therapy. In both sexes, but especially among the elderly, hidden blood loss via the gastrointestinal tract is also frequent as a result of benign diseases, iatrogenic complications, or neoplasia. Repeated blood donations may lead to iron deficiency. Reduced absorption may result from atrophic gastritis, chronic infection with Helicobacter pylori, celiac disease, often in atypical form, and may onset following duodenum bypass surgery or surgery resulting in the reduction of gastric acidity.

Iron deficiency is treated with iron administered orally, typically in doses corresponding to 100-200 mg of elemental iron. The most bioavailable form of iron is represented by ferrous salts, in particular, ferrous sulphate.

However, some patients are unable to tolerate oral treatment because of the side effects thereof, especially in the intestine (nausea, vomiting, constipation), or they have reduced compliance and must therefore be treated with parenteral iron. Chronic atrophic gastritis, which sometimes leads to H. pylori infection, also causes reduced absorption and said type of gastritis is often not reversible after eradication of the infection.

Recent studies suggest that poor tolerance of iron administered orally may be induced by unabsorbed iron, which could damage the intestinal mucosa due to the oxidising properties thereof. Furthermore, some studies suggest that excess iron in the intestine is harmful to the local flora, determining a change in the microbiota and the replacement of saprophytic species with pathogenic species.

There are also patients with poor absorption attributable to intestinal causes or treatment with pump inhibitors, which can be refractory to oral treatment.

One metal that is directly involved in the absorption and transport of iron is copper. Copper deficiency alters the role of other cellular constituents involved in antioxidant activities, such as iron, selenium, and glutathione, and consequently plays an important role in diseases in which oxidative stress is high and where there is reduced absorption of iron and impaired thyroid functioning.

The object of the present invention is therefore to provide a product which effectively treats iron deficiency, while also overcoming the drawbacks of oral iron administration.

### SUMMARY OF THE INVENTION

Said object has been achieved by an oral administration form comprising Fe(II) and Cu(II), as stated in claim 1.

In another aspect, the present invention relates to the use of said oral administration form for the treatment of iron deficiency.

In another aspect, the present invention relates to a unit dose of said oral administration form comprising Fe(II) and Cu(II).

In another aspect, the present invention relates to a food supplement comprising said oral administration form.

As will be clear by the following detailed description and working Examples provided for illustrative and non-limiting purposes, the oral administration form of the invention has surprisingly proved to be highly effective for supplementing iron in its most bioavailable form, namely Fe(II), while also offering optimal tolerability by the body, even over long periods of administration.

### BRIEF DESCRIPTION OF THE FIGURES

The characteristics and advantages of the present invention will become apparent from the following detailed description, working Examples provided for illustrative and non-limiting purposes and figures annexed hereto, wherein:

FIG. 1 shows the Fe content of formulations in the respective supernatant portions and pellets, according to Example 7,

FIG. 2 shows the concentration of Fe present in the apical (luminal) compartment at the beginning and at the end of the absorption step (3 hours, *p<0.05), according to Example 7, and

FIG. 3 shows the levels of ferritin present in the cells constituting the intestinal epithelium model following exposure to the three formulations in Example 7 (*p<0.05).

### DETAILED DESCRIPTION OF THE INVENTION

Therefore, the invention relates to an oral administration form comprising: a) an active core comprising a solid association of at least one pharmaceutically acceptable Fe(II) salt and at least one pharmaceutically acceptable Cu(II) salt and (b) an active core-coating layer, said coating layer comprising at least one enteric polymer and at least one hydrophilic polymer.

Divalent iron is the most bioavailable form because said form is absorbed by enterocytes. However, divalent iron irritates both the gastric mucosa and the intestinal mucosa. This irritation also has a negative effect on the absorption of said iron, to such an extent that long-term treatment is rendered ineffective.

It has unexpectedly been found that, by formulating the Fe(II) according to the present invention, it is possible to benefit from all the advantages of Fe(II) supplementation, but without experiencing the side effects stated above. Indeed, the coating layer effectively masks the typical taste of iron, offers long-term stability and, above all, prevents contact between the iron and the gastric walls, since it releases the iron directly within the enterocytes during fasting and onto the ileum in the small intestine.

At the same time, the possibility of using Fe(II) also overcomes the disadvantages associated with the use, as an alternative, of Fe(III), wherein said disadvantages usually manifest through an accumulation of iron in the liver. Indeed, the layer coating the oral administration form the invention also protects the Fe(II) present therein, advantageously preventing its oxidation to Fe(III).

Furthermore, the oral administration form is particularly stable as said form may be kept at room temperature for at least 24 months.

With the term “solid association”, it is understood that said at least one pharmaceutically acceptable Fe(II) salt and at least one pharmaceutically acceptable Cu(II) salt are mixed together to form a solid mixture or are present separately in the active core, both in the solid state. In fact, as explained in more detail when describing the preparation process, said salts may be pre-mixed together or added separately.

For the purposes of the present invention, suitable pharmaceutically acceptable salts of Fe(II) include both inorganic salts and complex compounds of Fe(II), as well as mixtures thereof.

Preferably, said at least one pharmaceutically acceptable Fe(II) salt is ferrous sulphate, ferrous fumarate, ferrous gluconate, ferrous succinate, ferrous glutamate, ferrous lactate, ferrous citrate, ferrous tartrate, ferrous pyrophosphate, ferrous carbonate, ferrous chloride, ferrous iodide, ferrous bisglycinate, ferrous saccharate, ferrous phosphate, ferrous formate, ferrous acetate, ferrous malate, ammonium ferrous sulphate, ferrous hydroxide, ferrous nitrate, ferrous oxalate, or a mixture thereof.

More preferably, said at least one pharmaceutically acceptable Fe(II) salt is ferrous fumarate, ferrous gluconate, ferrous succinate, ferrous lactate, ferrous citrate, ferrous tartrate, ferrous bisglycinate, ferrous oxalate, or a mixture thereof.

In particularly preferred embodiments, said at least one pharmaceutically acceptable Fe(II) salt is ferrous fumarate.

In other particularly preferred embodiments, said at least one pharmaceutically acceptable Fe(II) salt is in the form of micronised particles having a particle size distribution D₉₀ of 1-50 μm. For the purposes of the present invention, this parameter is measured through particle size analysis by sieving with suitable sieves arranged in series, each one of which retains the solid moiety whose granules are larger in size than the sieve holes. Preferably, said at least one pharmaceutically acceptable Fe(II) salt is in the form of micronised particles having a particle size distribution D₉₀ of 2-20 μm, more preferably 3-7 μm, and even more preferably of 5 μm.

For the purposes of the present invention, suitable pharmaceutically acceptable salts of Cu(II) include both inorganic salts and complex compounds of Cu(II), as well as mixtures thereof.

Preferably, said at least one pharmaceutically acceptable salt of Cu(II) is copper gluconate, copper citrate, copper lauroyl sarcosinate, copper formate, copper acetate, copper propionate, copper butyrate, copper lactate, copper oxalate, copper phytate, copper tartrate, copper malate, copper succinate, copper malonate, copper maleate, copper benzoate, copper salicylate, copper aspartate, copper glutamate, copper fumarate, copper glycerophosphate, chlorophyllin sodium copper, copper chloride, copper fluoride, copper sulphate, copper fluorosilicate, copper nitrate, copper fluoroborate, copper iodate, or a mixture thereof.

More preferably, said at least one pharmaceutically acceptable Cu(II) salt is copper gluconate, copper citrate, copper succinate, copper salicylate, copper aspartate, or a mixture thereof.

In particularly preferred embodiments, said at least one pharmaceutically acceptable Cu(II) salt is copper gluconate.

Preferably, in the oral administration form the invention, said at least one pharmaceutically acceptable Fe(II) salt is present in a greater amount than said at least one pharmaceutically acceptable Cu(II) salt.

In preferred embodiments of the invention, said at least one pharmaceutically acceptable Fe(II) salt and said at least one pharmaceutically acceptable Cu(II) salt are in a weight ratio of 200:1 to 2:1. Preferably, said at least one pharmaceutically acceptable Fe(II) salt and said at least one pharmaceutically acceptable Cu(II) salt are in a weight ratio of 100:1 to 10:1, more preferably 50:1 to 20:1.

In some embodiments, the oral administration form the invention comprises up to 10 wt 20% of said at least one pharmaceutically acceptable Fe(II) salt, preferably up to 5 wt %.

For the purposes of the present invention, unless otherwise specified, “wt %” means % of weight, based on the weight of the oral administration form of the invention.

In other embodiments, the oral administration form of the invention comprises up to 1 wt % of said at least one pharmaceutically acceptable Cu(II) salt, preferably up to 0.1 wt 25%.

The oral administration form of the invention can further comprise also pharmacologically acceptable excipients. The term “excipient” means a compound or a mixture thereof suitable for use in a formulation for the treatment of iron deficiency or conditions associated thereto. For example, an excipient for use in a pharmaceutical formulation should not generally cause an adverse response in a patient, nor should it significantly inhibit the efficacy of said formulation. Suitable excipients are diluents, disaggregating agents, glidants, binders, lubricants, stabilisers, adsorbents, release retardants, flavourings, sweeteners, anti-caking agents, and preservatives. In particular, they are used for the purposes of the present invention, natural starch, partially hydrolysed starch, lactose, glucose, sucralose, fructose, sucrose, mannitol, sorbitol, cellulose and derivatives thereof, microcrystalline cellulose and derivatives thereof, calcium phosphate, calcium carbonate, calcium sulphate, gelatin, tragacanth gum, gum arabic, polyethylene glycol, alginates, talc, silica, colloidal silica, precipitated silica, magnesium silicates, aluminium silicates, sodium lauryl sulphate, magnesium lauryl sulphate, methacrylate copolymers, potassium citrate tribasic monohydrate, polysorbate 20, polysorbate 40, polysorbate 60, polysorbate 80, esters of fatty acids, monoglycerides of fatty acids, diglycerides of fatty acids, and mixtures thereof.

Said at least one enteric polymer is a polymer capable of passing through the gastric tract, without, therefore, experiencing degradation in the presence of a strongly acidic pH), and then disintegrating once it has reached the intestinal tract with a basic pH, thus releasing the active ingredients contained therein.

Suitable enteric polymers are cellulose acetate phthalate, cellulose acetate succinate, hydroxypropyl methylcellulose phthalate, hydroxypropyl methylcellulose acetate succinate, polyvinyl acetate phthalate, hydroxyethyl cellulose phthalate, cellulose acetate tetrahydro phthalate, copolymers of methacrylate-methacrylic acid, copolymers of methyl methacrylate-methacrylic acid, sodium alginate, cellulose acetate trimellitate, shellac, or a mixture thereof.

Preferably, said at least one enteric polymer is shellac.

Said at least one hydrophilic polymer is a polymer capable of delaying the release of the active ingredients.

Suitable hydrophilic polymers are hydroxypropyl methylcellulose, hydroxypropyl cellulose, hydroxyethyl cellulose, xanthan gum, ethyl cellulose, cross-linked carboxy methyl cellulose and the salts thereof, polyvinyl alcohol, polymethacrylates regardless of the pH, polymethyl acetate, polyvinylpyrrolidone, or a mixture thereof.

Preferably, said at least one hydrophilic polymer is ethyl cellulose, hydroxypropyl cellulose, hydroxypropyl methylcellulose, polyvinylpyrrolidone, or a mixture thereof.

In preferred embodiments, said oral administration form furthermore comprises an inert agent selected from talc, corn starch, lactose, microcrystalline cellulose, polyethylene glycol, and mixtures thereof. Preferably, said oral administration form furthermore comprises talc and corn starch.

Preferably, said oral administration form is a capsule, tablet, mini-tablet, micro-tablet, granules, microgranules, pellet, multi-particulate, or micronised particulate.

Preferably, said oral administration form is orodispersible.

More preferably, said oral administration form is microgranules.

Preferably, said microgranules have a D₅₀ median particle-size distribution of 200-800 μm. For the purposes of the present invention, this parameter is measured through particle size analysis by sieving with suitable sieves arranged in series, each one of which retains the solid moiety whose granules are larger in size than the sieve holes.

In preferred embodiments, said microgranules have a D₅₀ median particle-size distribution of 250-600 μm.

The thickness of the coating layer is preferably 100-300 more preferably 100-200 and even more preferably of 100-500 μm.

The weight ratio of the active core to the coating layer is preferably of 6:1 to 1:1, more preferably 3:1 to 1:1, and even more preferably 2:1 to 1:1.

In other embodiments, the oral administration form of the invention further comprises at least one vitamin. Suitable vitamins are vitamin A, vitamin B1, vitamin B2, vitamin B6, vitamin B3, vitamin B9, vitamin B12, vitamin C, vitamin D, vitamin E, and vitamin K.

Preferably, the oral administration form of the invention further comprises at least vitamin C, since the latter facilitates the body's absorption of iron.

Preferably, the oral administration form of the invention comprises up to 15 wt % of vitamin C, and more preferably up to 10 wt %.

In some embodiments, the oral administration form of the invention further comprises folic acid, lipoic acid, lactoferrin or a mixture thereof.

In other embodiments, the oral administration form of the invention further comprises folic acid.

Preferably, the oral administration form of the invention comprises up to 0.2 wt % of folic acid, and more preferably up to 0.1 wt %.

In other embodiments, the oral administration form of the invention further comprises lipoic acid.

Preferably, the oral administration form of the invention comprises up to 15 wt % of lipoic acid, and more preferably up to 10 wt %.

In other embodiments, the oral administration form of the invention further comprises lactoferrin.

Preferably, the oral administration form of the invention comprises up to 15 wt % of folic acid, and more preferably up to 10 wt %.

In preferred embodiments, in the oral administration form of the invention, the active core a) comprises an internal core comprising at least one vitamin, coated with a first layer of at least one hydrophilic polymer and a second layer of a solid mixture of at least one pharmaceutically acceptable Fe(II) salt and at least one pharmaceutically acceptable Cu(II) salt. Preferably, the active core a) comprises an internal core comprising at least one vitamin, coated with a first layer of at least one hydrophilic polymer and an inert agent and a second layer of a solid mixture of at least one pharmaceutically acceptable Cu(II) salt and at least one pharmaceutically acceptable Fe(II) salt and at least one enteric polymer; and the coating layer b) comprises at least one enteric polymer, at least one hydrophilic polymer and at least one inert agent.

In other embodiments, in the oral administration form the invention, the active core a) comprises an internal core comprising at least one vitamin, coated with a first layer of at least one hydrophilic polymer, a second layer of at least one pharmaceutically acceptable Cu(II) salt, and a third layer of at least one pharmaceutically acceptable Fe(II) salt. Preferably, the active core a) comprises an internal core comprising at least one vitamin, coated with a first layer of at least one hydrophilic polymer and an inert agent, a second layer of at least one pharmaceutically acceptable Cu(II) salt, and a third layer of at least one pharmaceutically acceptable Fe(II) salt and at least one enteric polymer; and the coating layer b) comprises at least one enteric polymer, at least one hydrophilic polymer and at least one inert agent.

In further preferred embodiments, in the oral administration form the invention, the active core a) comprises an internal core comprising at least one vitamin, coated with a first layer comprising at least one hydrophilic polymer and at least one pharmaceutically acceptable Fe(II) salt, and a second layer comprising at least one hydrophilic polymer and at least one pharmaceutically acceptable Cu(II) salt. Preferably, the active core a) comprises an internal core comprising at least one vitamin, coated with a first layer comprising at least one hydrophilic polymer and at least one pharmaceutically acceptable Fe(II) salt, a second layer of an inert agent, and a third layer comprising at least one hydrophilic polymer and at least one pharmaceutically acceptable Cu(II) salt.

In other preferred embodiments, in the oral administration form the invention, the active core a) comprises an internal core comprising at least one vitamin, coated with a first layer comprising at least one hydrophilic polymer and at least one pharmaceutically acceptable Cu(II) salt, and a second layer comprising at least one hydrophilic polymer and at least one pharmaceutically acceptable Fe(II) salt. Preferably, the active core a) comprises an internal core comprising at least one vitamin, coated with a first layer comprising at least one hydrophilic polymer and at least one pharmaceutically acceptable Cu(II) salt, a second layer of an inert agent, and a third layer comprising at least one hydrophilic polymer and at least one pharmaceutically acceptable Fe(II) salt.

Preferably, said embodiments are in the form of microgranules.

It should be understood that the preferred aspects specified above for the individual components should likewise be considered as preferred also for these preferred embodiments.

In other embodiments, the oral administration form the invention does not comprise an internal core of inert agents.

In some embodiments, the oral administration form of the invention consists essentially of a) an active core comprising a solid association of at least one pharmaceutically acceptable Fe(II) salt and at least one pharmaceutically acceptable Cu(II) salt and optionally at least one vitamin, and (b) an active core-coating layer, said coating layer comprising at least one enteric polymer and at least one hydrophilic polymer. The expression “consists essentially of” means that said at least one pharmaceutically acceptable Fe(II) salt, said at least one pharmaceutically acceptable Cu(II) salt, and said optional at least one vitamin, are the only components present in the oral administration form of the invention which are active in the treatment of iron deficiency, the others being formulating components or pharmaceutically acceptable excipients which do not interfere with the action of Fe(II) and Cu(II).

In further embodiments, the oral administration form of the invention consists of a) an active core comprising a solid association of at least one pharmaceutically acceptable Fe(II) salt and at least one pharmaceutically acceptable Cu(II) salt and optionally at least one vitamin, and (b) an active core-coating layer, said coating layer comprising at least one enteric polymer and at least one hydrophilic polymer.

It should also be understood that all aspects identified as favourable and advantageous for the oral administration form of the invention should be deemed to be similarly preferable and advantageous also for these embodiments.

In preferred embodiments, the oral administration form of the invention is in the form of a unit dose.

Preferably, said unit dose comprises up to 200 mg of at least one pharmaceutically acceptable Fe(II) salt, more preferably 10-150 mg.

In particularly preferred embodiments, said unit dose comprises 50-100 mg of at least one pharmaceutically acceptable Fe(II) salt.

Preferably, said unit dose comprises up to 10 mg of at least one pharmaceutically acceptable CU(II) salt, more preferably 0.1-5 mg.

In particularly preferred embodiments, said unit dose comprises 0.5-2 mg of at least one pharmaceutically acceptable Cu(II) salt.

Preferably, said unit dose further comprises up to 300 mg of at least one vitamin, more preferably 50-200 mg.

Preferably, said unit dose further comprises 10-1500 μg of folic acid, more preferably 50-1000 μg.

In preferred embodiments, said unit dose comprises up to 200 mg of at least one pharmaceutically acceptable Fe(II) salt, up to 10 mg of at least one pharmaceutically acceptable Cu(II) salt, up to 300 mg of at least one vitamin, and optionally 10-1500 μg of folic acid.

In particularly preferred embodiments, said unit dose comprises up to 200 mg of Fe(II) fumarate, up to 10 mg of CU(II) gluconate, up to 300 mg of vitamin C, and 10-1500 μg of folic acid.

In particularly preferred embodiments, in said unit dose, the active core a) comprises an internal core comprising at least one vitamin, coated with a first layer of at least one hydrophilic polymer and a second layer of a solid association of at least one pharmaceutically acceptable Fe(II) salt and at least one pharmaceutically acceptable Cu(II) salt. Preferably, said unit dose embodiments are in the form of microgranules.

Particularly preferred are the unit dose embodiments in the form of microgranules, wherein a) the active core comprises an internal core comprising vitamin C, coated with a first layer of at least one hydrophilic polymer and a second layer of a solid association of Fe(II) fumarate and Cu(II) gluconate, and b) an active core-coating layer having a thickness of 100-300 μm, said coating layer comprising shellac and ethylcellulose, hydroxypropyl cellulose, hydroxypropyl methylcellulose, polyvinylpyrrolidone, or a mixture thereof.

It should be understood that the preferred aspects specified for the individual components should likewise be considered as preferred in the unit dose embodiments stated above.

The oral administration form of the present invention, as well as the unit dose thereof, may be prepared by methods known in the art.

In preferred embodiments, the oral administration form of the invention is obtained by a preparation process comprising the following steps:

i) providing a core comprising at least one vitamin,

ii) coating said core with a first layer of at least one hydrophilic polymer, optionally comprising at least one inert agent,

iii) coating the coated core from step ii) with a second layer of at least one pharmaceutically acceptable Fe(II) salt and at least one pharmaceutically acceptable Cu(II) salt, thus obtaining an active core, and

iv) coating the active core thus obtained with a coating layer comprising at least one enteric polymer and at least one hydrophilic polymer, optionally comprising at least one inert agent, thereby obtaining the oral administration form of the invention.

Preferably, said oral administration forms are microgranules, more preferably orodispersible microgranules.

Preferably, in step ii), said at least one hydrophilic polymer is dissolved in a hydroalcoholic solution before being applied to the core from step i).

Preferably, in step iii), said solid association is preliminarily dissolved in a suitable solvent and subsequently sprayed onto the core coated in step ii). In preferred embodiments, said at least one pharmaceutically acceptable Cu(II) salt applied prior to said at least one pharmaceutically acceptable Fe(II) salt. Preferably, said suitable solvent is an alcoholic solution of at least one enteric polymer.

Preferred solvents are ethanol and water-ethanol solutions.

In another aspect, the present invention relates to the use of the oral administration form described above for the treatment of iron deficiency. As mentioned, the oral administration form of the invention has, surprisingly, allowed to benefit from all the advantages of Fe(II) supplementation, but without experiencing the side effects stated above. Indeed, the coating layer effectively masks the typical taste of iron, is extremely stable over time, and above all prevents contact between iron and the gastric walls, since it releases the iron directly within the enterocytes.

Given the advantages found, in addition to the high tolerability by the body, the oral administration form of the invention is particularly recommended for iron supplementation in children, pregnant women, and the elderly.

In another aspect, the present invention relates to a food supplement comprising the oral administration form described above.

More preferably, the food supplement of the invention is administered orally.

In preferred embodiments, the food supplement of the invention is administered in doses of 1-5 g per day, said dose comprising up to 200 mg of at least one pharmaceutically acceptable Fe(II) salt.

The food supplement of the invention finds advantageous use in iron supplementation, because:
Fe(II) contributes to normal cognitive function, to normal energy metabolism, to the normal formation of red blood cells and haemoglobin, to the normal transportation of oxygen within the body, to the normal functioning of the immune system, to reducing tiredness and fatigue and to normal cognitive development in children, while intervening in the cell division process;
Cu(II) contributes to the maintenance of normal connective tissues, to normal energy metabolism, the normal functioning of the nervous system, normal pigmentation of hair, the normal iron transport in the body, normal pigmentation of skin, the normal functioning of the immune system, and the protection of cells from oxidative stress;
vitamin C, when present, increases absorption of iron, contributes to the normal formation of collagen for the normal function of blood vessels, to normal collagen formation for normal bone function, to normal collagen formation for normal cartilage function, to normal collagen formation for normal gingival function, normal collagen formation for normal skin function, normal training collagen for normal tooth function, to normal energy metabolism, to normal function of the nervous system, to normal psychological function, to normal function of the immune system, to the protection of cells from oxidative stress, the reduction of tiredness and fatigue, and to the regeneration of the reduced form of vitamin E.

It should be understood that all the possible combinations of the preferred aspects of the oral administration form, the unit doses, the preparation process, and the uses of said food supplement, the preparation process and the use of said oral administration form are disclosed herein and therefore are also preferred.

It should also be understood that all aspects identified as preferred and advantageous for the oral administration form and the components thereof, should be deemed to be similarly preferable and advantageous also for the uses of said food supplement, and the preparation processes and uses of said oral administration form.

Below are working examples of the present invention provided for illustrative purposes.

### EXAMPLES

### Example 1

A 1.5 g oral administration form was prepared comprising iron fumarate (wherein 30 mg Fe(II)), copper gluconate (wherein 1 mg Cu(II)), 80 mg vitamin C, talc, fructose, flavouring agent, sucralose, silicon dioxide, shellac, hydroxypropyl cellulose, and hydroxypropyl methylcellulose.

A vitamin C core was prepared, which was subsequently coated with a first layer of hydroxypropyl methylcellulose and talc, starting from a water-ethanol solution.

A second layer was applied to the core thus coated by spraying copper gluconate in ethanol and shellac.

A third layer was then applied by spraying iron fumarate in ethanol and shellac.

In this manner, the active core was obtained.

Finally, said active core was coated with a coating layer comprising shellac, hydroxypropyl cellulose, and talc, thus obtaining the oral administration form of the invention.

### Example 2

The oral administration form in Example 1 was subjected to an in vitro test called “Dissolution tests for solid oral forms” according to the European Pharmacopoeia, to assess the release of Fe(II):

| Analysis | Result |
|---|---|
| In HCl 0.1M solution: | |
| after 0.5 h | 31.9% |
| after 1 h | 54.9% |
| after 2 h | 58.6% |
| In a pH 6.8 solution: | |
| after 3 h | 53.4% |
| In a pH 8 solution: | |
| after 2 h | >82% |

### Example 3

A 1.5 g oral administration form was prepared comprising iron fumarate (wherein 30 mg Fe(II)), copper gluconate (wherein 1 mg Cu(II)), 80 mg vitamin C, 400 μg folic acid, talc, fructose, flavouring agent, sucralose, silicon dioxide, shellac, hydroxypropyl cellulose, and hydroxypropyl methylcellulose.

A vitamin C core was prepared, which was subsequently coated with a first layer of hydroxypropyl methylcellulose and talc, starting from a water-ethanol solution.

A second layer was applied to the core thus coated by spraying copper gluconate, iron fumarate, and folic acid in ethanol and shellac.

In this manner, the active core was obtained.

Finally, said active core was coated with a coating layer comprising shellac, hydroxypropyl cellulose, and talc, thus obtaining the oral administration form of the invention.

### Example 4

A 1.5 g oral administration form was prepared comprising iron fumarate (wherein 40 mg Fe(II)), copper gluconate (wherein 0.1 mg Cu(II)), 20 mg vitamin C, fructose, flavouring agent, sucralose and silicon dioxide.

The layer of coating comprised ethyl cellulose and cellulose acetate succinate, thus obtaining the oral administration form of the invention, according to the procedure in Example 1.

### Example 5

A 1.5 g oral administration form was prepared comprising iron oxalate (wherein 35 mg Fe(II)), copper aspartate (wherein 2 mg Cu(II)), 100 mg vitamin C, corn starch, fructose, flavouring, sucralose, silicon dioxide, shellac, hydroxymethyl cellulose and hydroxypropyl methylcellulose.

A vitamin C core was prepared, which was subsequently coated with a first layer of hydroxymethyl cellulose and corn starch, starting from a water-ethanol solution.

A second layer was applied to the core thus coated by spraying copper gluconate and iron fumarate in ethanol, with hydroxymethyl cellulose and hydroxypropyl methylcellulose.

In this manner, the active core was obtained.

Finally, said active core was coated with a coating layer comprising shellac, hydroxypropyl cellulose, and talc, thus obtaining the oral administration form of the invention.

### Example 6

A 1.5 g oral administration form was prepared comprising iron lactate (wherein 35 mg Fe(II)), copper succinate (wherein 2 mg Cu(II)), 100 mg vitamin C, fructose, flavouring agent, sucralose, and silicon dioxide.

The layer of coating comprised shellac and hydroxypropyl methylcellulose, thus obtaining the oral administration form of the invention, according to the procedure in Example 3.

### Example 7

A 1.5 g oral administration form was prepared comprising iron fumarate (wherein 30 mg Fe(II)), copper gluconate (wherein 1 mg Cu(II)), 80 mg vitamin C, talc, shellac, hydroxypropyl cellulose, and hydroxypropyl methylcellulose.

A vitamin C core was prepared, which was subsequently coated with a first layer of hydroxypropyl methylcellulose, iron fumarate, and talc, starting from a water-ethanol solution.

A second layer was applied to the core thus coated by spraying copper gluconate in ethanol and shellac.

In this manner, the active core was obtained.

Finally, said active core was coated with a coating layer comprising shellac, hydroxypropyl cellulose, and talc, thus obtaining the oral administration form of the invention.

### Example 8

Comparative Assessment of Intestinal Iron Absorption

A study was carried out in order to assess intestinal and intracellular absorption of the iron released by oral administration forms of the present invention, comparing said absorption with that of comparative formulations.

To this end, the following formulations were provided:

1) granular administration form of Example 7 of the invention (shortly: Invention Granule),

2) granular administration form of Example 7 but without copper gluconate (shortly: Control Granule),

3) solid mixture of the ingredients listed in Example 7 (shortly: Mixture).

In order to assess Fe release, formulations 1-3 were treated as follows: equal amounts of the different formulations were resuspended in hydrochloric acid (HCl) 0.1 M (pH 1.2) and left under stirring for 2 hours at 37° C.; next, the different suspensions were diluted in culture medium devoid of FBS (Fetal Bovine Serum) and, after neutralising the pH thereof (final pH: 6.8), were left under stirring at 37° C. for a further 3 hours.

In Vitro Model of Intestinal Epithelium

Absorption of Fe was determined by using an in vitro model of human intestinal epithelium based on human Caco-2 intestinal adenocarcinoma cells, organised as a functional monolayer on Transwell® inserts. Briefly, Transwell® inserts featured two compartments, apical (or luminal) and basolateral (or serosal), separated by a microporous membrane. Caco-2 monolayers cultivated on microporous membranes were made up of polarised cells with the typical morpho-functional features of enterocytes, such as the presence of microvilli, occluding junctions (tight junctions) and glycoprotein-p (P-gp).

Assessment of Intestinal Absorption of Fe

The bioaccessible fractions of formulations 1-3 were added to the apical compartment of the in vitro intestinal epithelium, while the FBS-free cellular medium was added to the basolateral compartment. After 3 hours of incubation, the apical fractions and the Caco-2 cell monolayers were recovered and the respective Fe content determined by ICP-MS analysis.

Determination of Intracellular Ferritin

At the end of the 3 hours of exposure, the Caco-2 cell monolayers were subjected to repeated washings in order to remove the unabsorbed Fe, and left to incubate for a further 21 hours in cellular medium (1% FBS) [Scheers N M, et al., “Proposing a Caco-2/HepG2 cell model for in vitro ironabsorption studies”, J NutrBiochem., 2014], in order to allow expression of ferritin, a protein involved in the storage of Fe. Once incubation has ended, the Caco-2 cells were resuspended by trypsin-EDTA treatment, pelleted and sonicated in an appropriate buffer. The ferritin content of the various cellular lysates was determined by ELISA (Enzyme-LinkedImmunoSorbentAssay), using a commercial kit.

Results

Release of Fe from the Three Formulations—“Bio-Accessible Fractions”

At the end of the treatment step disclosed above (incubation of the three formulations in HCl and subsequent pH neutralisation), the total Fe concentration was similar among Formulations 1-3 and compatible with the expected formulation (approx. 1200 ppm).

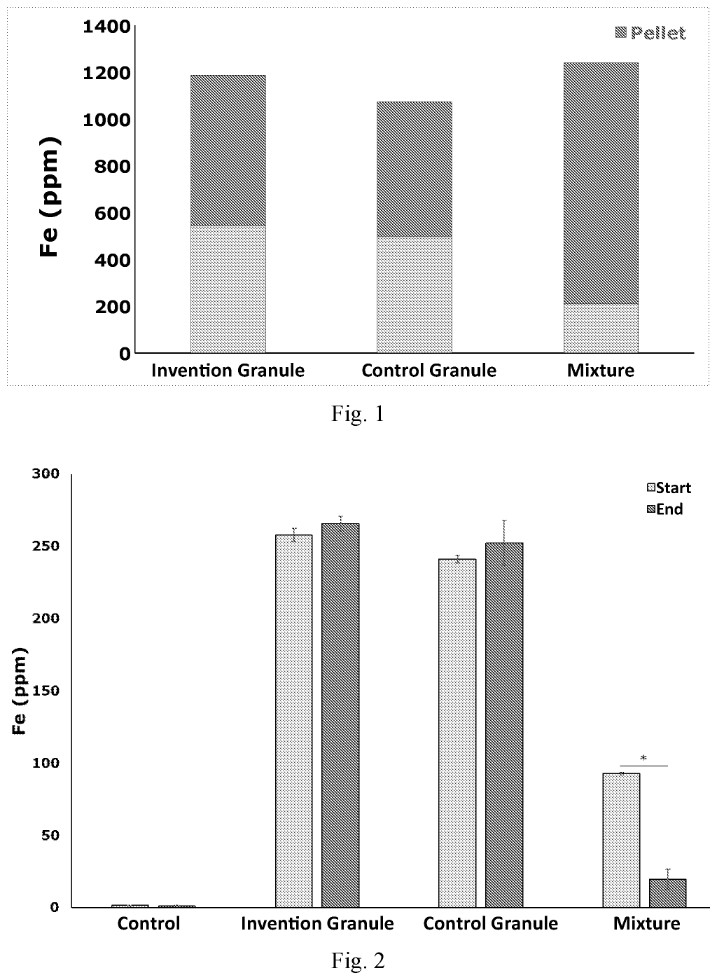

As illustrated in FIG. 1, most of the Fe present in the Mixture formulation is located in the pellets (1030.6 ppm corresponding to 83.1% of total Fe), indicating a poor propensity of the mixture to release Fe in a bio-accessible form (only 16.9% of total Fe was present in the supernatant).

The Control Granule formulation has a concentration of Fe in the supernatant of 498.8 ppm, indicating that this form of administration promotes the release of Fe.

Surprisingly, the Invention Granule formulation further increased the concentration of Fe in the supernatant, since a value of 544.1 ppm was observed, i.e. an increase of approximately 10% compared to the formulation in granules without Cu.

Intestinal Absorption of Fe

In order to assess Fe absorption, intestinal epithelia were exposed for 3 hours to the bio-accessible fractions of Formulations 1-3. At the end of the exposure period, Fe levels were monitored in the apical (lumen) and intracellular fractions.

As can be seen from FIG. 2, the bio-accessible fraction of Fe derived from the Mixture showed a significant drop in apical (luminal) Fe during period of exposure, mainly due to precipitation phenomena.

Differently, the bio-accessible fractions of granule formulations are stable over time, even if the Invention Granule fraction resulted to be more stable.

Intracellular absorption of Fe released from the three formulations was assessed by determining the quantity of micronutrient and ferritin in the enterocytes. More specifically, ferritin was selected as an indicator of intracellular iron content as involved in the Fe storage process and its expression depends on intracellular Fe levels.

The intracellular Fe concentration of the Invention Granule and Control Granule formulations was approximately 10 times greater than in the untreated control. The amount of Fe measured in the intestinal epithelium, however, was not representative of the actual intracellular content of the micronutrient, due to the precipitation phenomenon described above, which leads to an aspecific, hard-to-remove Fe deposit.

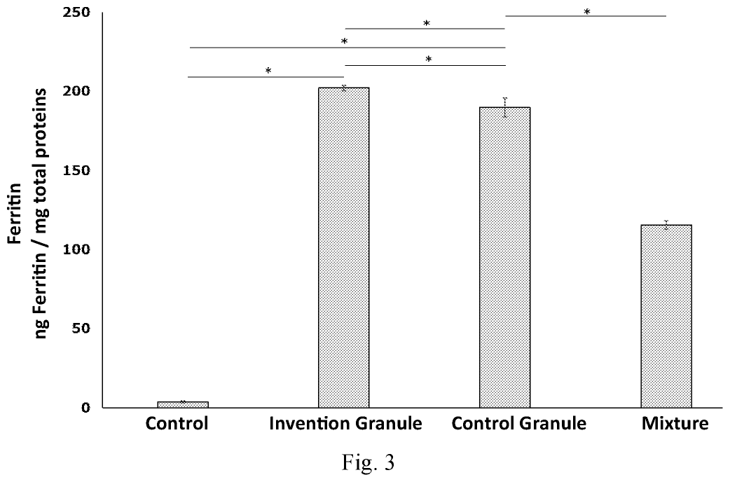

Indeed, contrary to expectations from the intracellular Fe concentration data obtained, while the ferritin induced following exposure to the Mixture was 115 ng ferritin/mg total protein (FIG. 3), the cells treated with the Control Granule formulation showed ferritin levels of 189 ng ferritin/mg protein total and, most surprisingly, cells treated with the Invention Granule formulation had ferritin levels of 202 ng ferritin/mg total protein, i.e. approximately 7% higher than the levels observed for the Control Granule.

Reduced expression of ferritin in cells treated with the Mixture formulation indicates that the amount of Fe actually absorbed at cellular level is lower than with the Control Granule formulation, and even more so than with the Invention Granule formulation. As can be seen from this data, the Invention Granule leads to a significant increase with respect to the Control Granule and the Mixture. Such difference is attributed to the presence of copper (Cu), which assists cellular intake of Fe.

Based on the foregoing, the oral administration form of the present invention has proved to be capable of guaranteeing a much higher iron release than the formulation in a simple solid mixture, demonstrating the fact that, upon use of the same composition, the combination of active core and coating layer of the invention is crucial. Furthermore, the presence of copper proved to be even more crucial in increasing intracellular iron absorption compared to the copper-free formulation, as confirmed by the higher ferritin levels present in cells treated with the Invention Granule formulation.

1. An oral administration form comprising: a) an active core comprising a solid association of at least one pharmaceutically acceptable Fe(II) salt and at least one pharmaceutically acceptable Cu(II) salt, and (b) an active core-coating layer, said coating layer comprising at least one enteric polymer and at least one hydrophilic polymer.

2. The oral administration form of claim 1, wherein said at least one pharmaceutically acceptable Fe(II) salt is ferrous fumarate, ferrous gluconate, ferrous succinate, ferrous lactate, ferrous citrate, ferrous tartrate, ferrous bisglycinate, ferrous oxalate, or a mixture thereof.

3. The oral administration form of claim 1, wherein said at least one pharmaceutically acceptable Cu(II) salt is copper gluconate, copper citrate, copper succinate, copper salicylate, copper aspartate, or a mixture thereof.

4. The oral administration form of claim 1, wherein said at least one pharmaceutically acceptable Fe(II) salt is present in a greater amount than said at least one pharmaceutically acceptable Cu(II) salt.

5. The oral administration form of claim 1, wherein the oral administration form comprises up to 10 wt % of said at least one pharmaceutically acceptable Fe(II) salt.

6. The oral administration form of claim 1, wherein the oral administration form comprises up to 1 wt % of said at least one pharmaceutically acceptable Cu(II) salt.

7. The oral administration form of claim 1, wherein the thickness of the coating layer b) is 100-300 μm, and the weight ratio of the active core to the coating layer is of 6:1 to 1:1.

8. The oral administration form of claim 1, further comprising at least one vitamin.

9. The oral administration form of claim 8, wherein the active core a) comprises an internal core comprising at least one vitamin coated with a first layer of at least one hydrophilic polymer and a second layer of a solid mixture of at least one pharmaceutically acceptable Fe(II) salt and at least one pharmaceutically acceptable Cu(II) salt.

10. The oral administration form of claim 8, wherein the active core a) comprises an internal core comprising at least one vitamin, coated with a first layer of at least one hydrophilic polymer, a second layer of at least one pharmaceutically acceptable Cu(II) salt, and a third layer of at least one pharmaceutically acceptable Fe(II) salt.

11. The oral administration form of claim 8, wherein the active core a) comprises an internal core comprising at least one vitamin, coated with a first layer comprising at least one hydrophilic polymer and at least one pharmaceutically acceptable Fe(II) salt, and a second layer comprising at least one hydrophilic polymer and at least one pharmaceutically acceptable Cu(II) salt.

12. The oral administration form of claim 8, wherein the active core a) comprises an internal core comprising at least one vitamin, coated with a first layer comprising at least one hydrophilic polymer and at least one pharmaceutically acceptable Fe(II) salt, a second layer of an inert agent, and a third layer comprising at least one hydrophilic polymer and at least one pharmaceutically acceptable Cu(II) salt.

13. The oral administration form of claim 8, in the form of a unit dose comprising up to 200 mg of at least one pharmaceutically acceptable Fe(II) salt, up to 10 mg of at least one pharmaceutically acceptable Cu(II) salt, up to 300 mg of at least one vitamin, and optionally 10-1500 μg of folic acid, said unit dose being preferably in the form of microgranules.

14. A method of treating iron deficiency, said method comprising the step of administering to a subject in need thereof the oral administration form of claim 1.

15. A food supplement comprising the oral administration form of claim 1.

16. The oral administration form of claim 1, wherein said at least one pharmaceutically acceptable Fe(II) salt is ferrous fumarate, and wherein said at least one pharmaceutically acceptable Cu(II) salt is copper gluconate.

17. The oral administration form of claim 1, wherein said at least one pharmaceutically acceptable Fe(II) salt and said at least one pharmaceutically acceptable Cu(II) salt are in a weight ratio of 100:1 to 10:1.

18. The oral administration form of claim 17, wherein said at least one pharmaceutically acceptable Fe(II) salt and said at least one pharmaceutically acceptable Cu(II) salt are in a weight ratio of 50:1 to 20:1.

19. The oral administration form of claim 1, wherein said oral administration form comprises up to 5 wt % of said at least one pharmaceutically acceptable Fe(II) salt, and up to 0.1 wt % of said at least one pharmaceutically acceptable Cu(II) salt.

20. The oral administration form of claim 7, wherein the weight ratio of the active core to the coating layer is of 3:1 to 1:1.
